(19) United States
(12) Patent Application Publication
(10) Pub. No.: US 2008/0118955 A1
(43) Pub. Date: May 22, 2008

(54) METHOD FOR PRECISE TEMPERATURE CYCLING IN CHEMICAL / BIOCHEMICAL PROCESSES

(75) Inventors: Siddhartha Panda, Kanpur (IN); Richard S. Wise, Newburgh, NY (US)

Correspondence Address: CANTOR COLBURN LLP - IBM FISHKILL, 20 Church Street, 22nd Floor, Hartford, CT, 06103, US

(73) Assignee: INTERNATIONAL BUSINESS MACHINES CORPORATION, Armonk, NY (US)

(21) Appl. No.: 11/858,280

(22) Filed: Sep. 20, 2007

Related U.S. Application Data
(63) Continuation-in-part of application No. 11/307,936, filed on Feb. 28, 2006, now abandoned.
Application No. 11/307,936 is a division of application No. 10/709,318, filed on Apr. 28, 2004, now abandoned.

(51) Int. Cl. C12P 19/34; B01J 19/12

(52) U.S. Cl. 435/91.2; 204/157.15

(57) ABSTRACT

A method for implementing a temperature cycling operation for a biochemical sample to be reacted includes applying an infrared (IR) heating source to the biochemical sample to be reacted at a first infrared wavelength selected so as to generate a first desired temperature for a first duration and produce a first desired reaction within the biochemical sample; following the first desired reaction, applying the infrared (IR) heating source to the biochemical sample at a second infrared wavelength selected so as to generate a second desired temperature for a second duration and produce a second desired reaction within the biochemical sample; and wherein the first and second wavelengths generated by the IR source are selected to be coincident with corresponding absorptive wavelengths of the biochemical sample so as to heat the biochemical sample without directly heating a fluid medium containing the biochemical sample.

### CROSS REFERENCE TO RELATED APPLICATIONS

This is a continuation-in-part application of U.S. patent application Ser. No. 11/307,936, filed Feb. 28, 2006, which is in turn a divisional application of U.S. patent application Ser. No. 10/709,318, entitled “METHOD AND APPARATUS FOR PRECISE TEMPERATURE CYCLING IN CHAMICAL/BIOCHEMICAL PROCESSES,” filed Apr. 28, 2004, now abandoned, which is incorporated herein by reference.

### BACKGROUND

The present invention relates generally to temperature control systems, and, more particularly, to a method for precise temperature cycling in chemical/biochemical processes, such as nucleic acid amplification, DNA sequencing and the like.

Polymerase Chain Reaction (PCR) is a chemical amplification technique developed in 1985 by Kary Mullis, in which millions of copies of a single DNA fragment may be replicated for use in research or forensic analysis. PCR involves three basic steps, each of which is performed at a specific temperature. To be most effective, these temperature changes should be as rapid as possible. In the first step, denaturing, a test tube containing the fragment is heated to about 95° C. for a few seconds, thereby causing the double-stranded DNA fragment to separate into two single strands. The second step is annealing, in which the temperature of the test tube is then lowered to about 55° C. for a few seconds, causing primers to bind permanently to their sites on the single-stranded DNA. The third step is extending, in which the temperature is raised to about 72° C. for about a minute, which causes the polymerase protein to go to work.

The protein moves along the single-stranded portion of the DNA, beginning at a primer, and creates a second strand of new DNA to match the first. After extension, the DNA of interest is double-stranded again, and the number of strands bearing the sequence of interest has been doubled. These three steps are then repeated about 30 times, resulting in an exponential increase of up to a billion-fold of the DNA of interest. Thus, a fragment of DNA that accounted for one part in three million, for example, now fills the entire test tube.

In conventional PCR equipment, an array of tubes or vials holding samples of DNA is placed in a metal block, and the temperature of the samples is controlled by heating and cooling the block. An alternative apparatus involves the use of a rapid thermal cycler, wherein samples are placed in a plastic plate having water circulating underneath to set the temperature of the samples. In order to change the temperature of the samples in such a device, water is switched from one tank to another.

However one disadvantage of such existing PCR heating devices is the large thermal budget needed to heat the metal block or water. In addition, precise temperature control issues may also present a problem in that physical heat transfer mechanisms (e.g., conduction, convection) are needed to transfer heat from the metal block/water to the container, and then to the cultures themselves. Still another concern related to conventional heating equipment relates to the lag time associated with a change in temperature settings.

Accordingly, it would be desirable to implement a more precise heating system for chemical and biochemical uses, such as performing PCR.

### SUMMARY

The foregoing discussed drawbacks and deficiencies of the prior art are overcome or alleviated, in an exemplary embodiment, by a method for implementing a temperature cycling operation for a biochemical sample to be reacted, including applying an infrared (IR) heating source to the biochemical sample to be reacted at a first infrared wavelength selected so as to generate a first desired temperature for a first duration and produce a first desired reaction within the biochemical sample; following the first desired reaction, applying the infrared (IR) heating source to the biochemical sample at a second infrared wavelength selected so as to generate a second desired temperature for a second duration and produce a second desired reaction within the biochemical sample; and wherein the first and second wavelengths generated by the IR source are selected to be coincident with corresponding absorptive wavelengths of the biochemical sample so as to heat the biochemical sample without directly heating a fluid medium containing the biochemical sample.

In another embodiment, a method for implementing temperature cycling for a polymerase chain reaction (PCR) process includes inserting a DNA fragment into an infrared (IR) reaction chamber; activating an infrared (IR) heating source within the reaction chamber at a first infrared wavelength selected so as to generate within the DNA fragment a first temperature for a first duration until a denaturing step is completed; following the denaturing step, activating the infrared (IR) heating source at a second infrared wavelength selected so as to generate within the DNA fragment a second temperature for a second duration until an annealing step is completed; and following the annealing step, activating the infrared (IR) heating source at a third infrared wavelength selected so as to generate within the DNA fragment a third temperature for a third duration until an extending step is completed; wherein the first, second and third wavelengths generated by the IR source are selected to be coincident with corresponding absorptive wavelengths of the DNA fragment without being coincident with corresponding absorptive wavelengths of a fluid medium containing the DNA fragment so as to avoid so as to heat the DNA fragment without directly heating the fluid medium.

### BRIEF DESCRIPTION OF DRAWINGS

Referring to the exemplary drawings wherein like elements are numbered alike in the several Figures:

FIG. 1 is a schematic illustration of a resonant, infrared reaction chamber, suitable for use in accordance with an embodiment of the invention;

FIG. 2(a) is a graph illustrating a method for implementing a temperature cycling operation for a biochemical sample to be reacted, in accordance with an embodiment of the invention;

FIG. 2(b) is a graph illustrating a method for implementing a temperature cycling operation for a biochemical sample to be reacted, in accordance with an alternative embodiment of the invention;

FIG. 3 is a schematic illustration of a method for implementing a continuous, temperature cycling batch operation for a biochemical sample to be reacted, in accordance with still another embodiment of the invention; and

FIG. 4 is a graph depicting molecular absorptivity of water and other materials as a function of wavelength.

### DETAILED DESCRIPTION

Disclosed herein is a method and apparatus for precise temperature cycling in chemical/biochemical processes (e.g., PCR), in which infrared (IR) resonant heating is used to selectively heat a chemical/biochemical culture. When electromagnetic (EM) radiation resonates at the natural vibrational frequency of a bond of a molecule in the material to which the EM energy is applied, the energy is absorbed and is manifested as heating, as a result of an increased amplitude of vibration. The resonant heating therefore enhances specificity of reactions, since only the desired molecules are directly heated by application of specific wavelengths of the EM radiation. With a large number of vibrational modes available for any given asymmetric surface species, resonance at a specific IR wavelength can be exploited to heat only the desired component. As a result, the application of selective resonant heating can effectively heat specific bonds to a desired temperature, thus attaining a much higher desired fractional dissociation relative to existing heating mechanism, without undesirable side reactions.

Moreover, since IR radiation heats the biochemical samples without directly heating the fluid medium carrying the samples, this results in a fast, one-stage heat transfer that can conceivably lower the PCR cycle time from about 2-3 minutes, to possibly to a few seconds. Furthermore, since only the bonds of interest are activated by the IR radiation, the effects of heating a metal/fluid or sample vials do not come into play, thereby lowering the overall thermal budget.

Although the embodiments described hereinafter are presented in the context of the PCR process, it should be appreciated that this process has been chosen herein as just one example to highlight the advantages of the IR resonant heating methodology. As such, the present invention embodiments are not to be construed as being specifically limited to the PCR process, but rather can be applied to a broad range of chemical/biochemical systems and processes. As used herein, the term “sample” refers to the specimen (e.g., organic compound, DNA fragment) that is to be heated so as to result in a desired chemical reaction of the specimen. A sample “medium” refers to a fluid medium that contains the specimen to be reacted. Although “medium” may also generally refer to components such as specimen vials or holding blocks. A “fluid medium” is the fluid in which the sample/specimen to be reacted is contained.

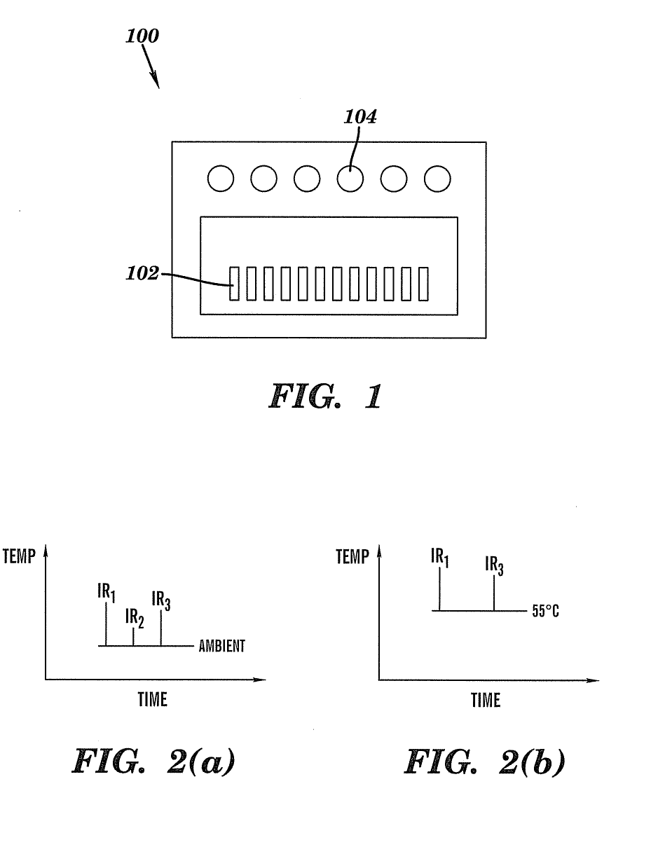

Referring initially to FIG. 1, there is shown a schematic illustration of a resonant, infrared reaction chamber 100, suitable for use in accordance with an embodiment of the invention. The chamber 100 is configured to receive a plurality of specimen vials 102 therein, such as DNA fragment containing test tubes for PCR amplification, for example. A plurality of infrared radiation generation sources 104 are also included for providing EM radiation at one or more specifically desired wavelengths, such as in the Near IR or Mid IR bands. The IR sources may be obtained from any commercially available source, and preferably provide a broad range of spectral radiance (e.g., 1-1000 W/cm²).

In a temperature cycling process, such as the three-step process involved in PCR, the chamber 100 is configured to apply specifically targeted IR wavelengths to the vial contents in order to produce the three distinct reactions that take place at the different temperature values specified above. Thus, as shown in FIG. 2(a), once the vials are placed within the chamber 100 (at about ambient temperature), they are initially subjected to a first IR wavelength (IR1) specifically selected to carry out the denaturing step at about 95° C. for about 30 seconds to separate the DNA into single strands. Then, the samples are subjected to a second IR wavelength (IR2) specifically selected to carry out the annealing step at about 55° C. for about 30 seconds for the primers to bind to the sites on the single strands. Finally, the samples are subjected to a third IR wavelength (IR3) specifically selected to carry out the extending step at about 75° C. for about a minute, where the polymerase protein creates new DNA to match the original.

In an alternative embodiment, a three-step temperature cycling process may be performed using two IR energy wavelengths. As depicted by the graph in FIG. 2(b), the process chamber is initially heated and kept at a temperature representing the lowest of the three desired temperature values (in this example, 55° C.). Thus, to implement the PCR process, the vials are initially subjected to the first IR wavelength (IR1) for denaturing. Then, because the chamber is already heated to a baseline temperature of 55° C., no IR radiation is applied for a duration representing the completion time of the annealing step. In other words, the second IR wavelength (IR2) used in the embodiment of FIG. 2(a) is not used. Then, after the vials are exposed to the preheated annealing temperature for the requisite time, third IR wavelength (IR3) is applied to the vials for the extending step.

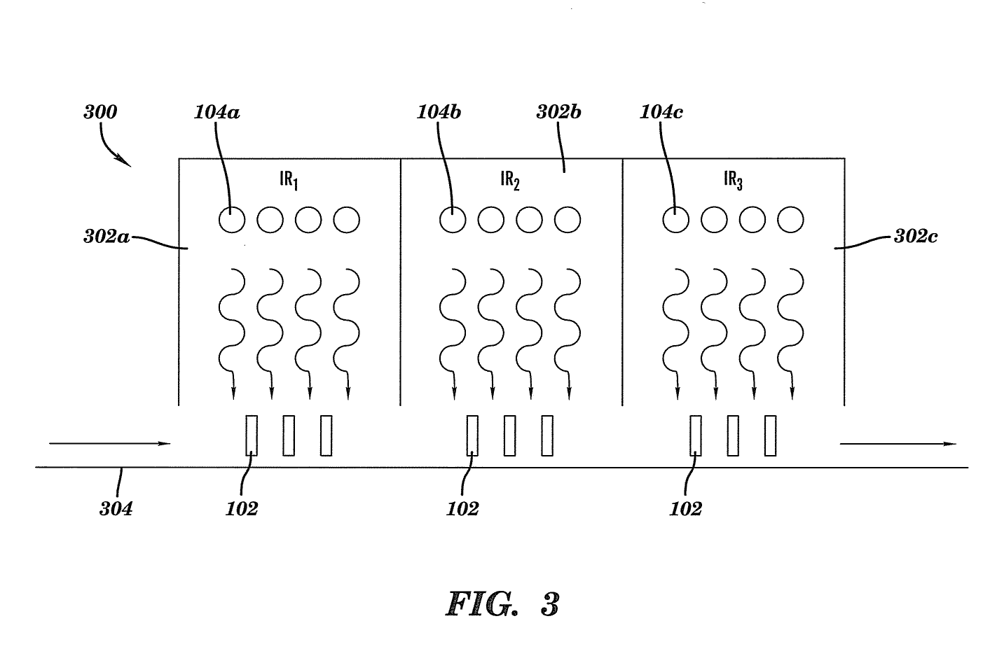

Still a further embodiment of a precise temperature cycling method and apparatus is shown in FIG. 3. As is shown, the system 300 can also be designed to conduct a batch operation in a continuous mode. Instead of using a single processing chamber with an infrared heating source of varying wavelengths, the samples 102 are exposed to IR radiation at specified wavelengths in discrete chambers 302a, 302b, 302c, by traveling along conveyor 304. Again, using the PCR example, the first chamber will include IR generation sources 104a configured for directing IR energy at the first IR wavelength (IR1); the second chamber will include IR generation sources 104b configured for directing IR energy at the second IR wavelength (IR2); and the third chamber will include IR generation sources 104c configured for directing IR energy at the third IR wavelength (IR3). This embodiment thus allows for higher throughput as the industry prepares to meet growing needs in the near future.

As will be appreciated from the above described embodiments, certain disadvantages of existing thermal cyclers used in the art (e.g., such as those having sample vials of DNA placed in either a metal block or in wells in a plastic plate with circulating fluid) are overcome, since the temperature of the samples is not controlled by the temperature of a metal block or circulating heating oil. As a result, thermal resistance issues emanating from conductive/convective heat transfer from a metal/fluid to polypropylene vials and then to the sample are avoided by the use of IR resonant heating.

Sample throughput may thus be increased due to a decreased lag time as a result of the time needed to change the cycle temperature settings in view of thermal resistances. Furthermore, the above described embodiments can alleviate the possibility of cross-reactivity with non-targeted DNA sequencing that could otherwise result in non-specific amplification and primers reacting with one other.

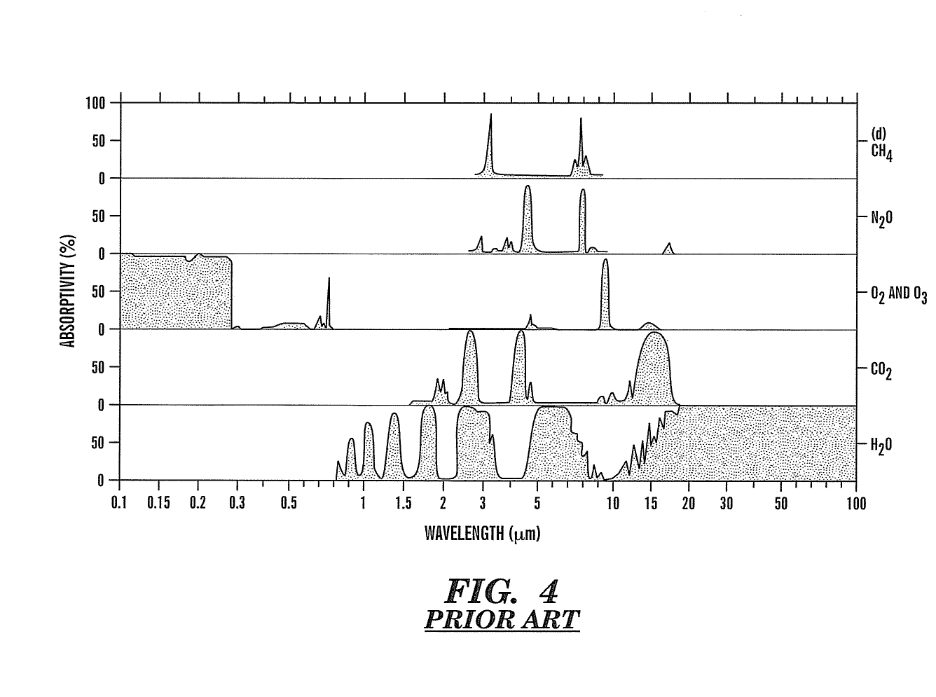

FIG. 4 is a graph depicting molecular absorptivity of water and other materials as a function of wavelength. As can be seen from the bottom portion of the graph, there are several pockets of wavelength ranges within the IR and near IR spectra in which there is no IR absorption by water. These ranges include: about 8.5-10 μm (1000-1200 cm⁻¹); about 3.6-4.2 μm (2400-2800 cm⁻¹); about 2.0-2.4 μm (4200-5000 cm⁻¹); about 1.5-1.7 μm (5880-6600 cm⁻¹); and about 1.2 μm (8333 cm⁻¹). Thus, at applied IR wavelengths in these ranges, any organic material (contained in water) having a natural vibrational frequency of a bond that falls therein will be subject to resonant heating but without causing resonant heating of the water as well.

Finally, Table 1 below lists some exemplary organic compounds that have a natural vibrational frequency of a bond of a molecule that falls within one of the wavelength ranges in which water does not absorb IR. Thus, such compounds may be directly heated by IR radiation in this frequency without directly heating the fluid medium (water) that contains the biochemical sample.

TABLE 1

| Frequency | | |
|---|---|---|
| (cm⁻¹) | Vibration | Compound |
| 1130-1100 | Symmetric C═C═C stretch (2 bands) | Allenes |
| 1130 | Pseudosymmetric C═C═O stretch | Ketene |
| 1065 | C═S stretch | Ethylene |
| | | trithiocarbonate |

While the invention has been described with reference to a preferred embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method for implementing a temperature cycling operation for a biochemical sample to be reacted, the method comprising:
applying an infrared (IR) heating source to the biochemical sample to be reacted at a first infrared wavelength selected so as to generate a first desired temperature for a first duration and produce a first desired reaction within the biochemical sample;
following the first desired reaction, applying the infrared (IR) heating source to the biochemical sample at a second infrared wavelength selected so as to generate a second desired temperature for a second duration and produce a second desired reaction within the biochemical sample; and
wherein the first and second wavelengths generated by the IR source are selected to be coincident with corresponding absorptive wavelengths of the biochemical sample so as to heat the biochemical sample without directly heating a fluid medium containing the biochemical sample.

2. The method of claim 1, further comprising:
following the second desired reaction, applying the infrared (IR) heating source to the biochemical sample at a third infrared wavelength selected so as to generate a third desired temperature for a third duration and produce a third desired reaction within the biochemical sample, wherein the third wavelength generated by the IR source is selected to be coincident with a corresponding absorptive wavelength of the sample so as to heat the biochemical sample without directly heating the fluid medium containing the sample.

3. The method of claim 2, wherein the biochemical sample is placed within a reaction chamber during the application of each of the infrared (IR) heating source at each of the first, the second and the third wavelengths.

4. The method of claim 2, further comprising:
passing the biochemical sample through a first chamber, the first chamber having the first infrared wavelength generated therein;
passing the biochemical sample through a second chamber, the second chamber having the second infrared wavelength generated therein; and
passing the sample through a third chamber, the third chamber having the third infrared wavelength generated therein.

5. The method of claim 4, wherein the biochemical sample is passed through the first second and third chambers by a conveyor.

6. The method of claim 1, wherein the first and second wavelengths correspond to a frequency range of about 1000 cm⁻¹ to about 1200 cm⁻¹.

7. A method for implementing temperature cycling for a polymerase chain reaction (PCR) process, the method comprising:
inserting a DNA fragment into an infrared (IR) reaction chamber;
activating an infrared (IR) heating source within the reaction chamber at a first infrared wavelength selected so as to generate within the DNA fragment a first temperature for a first duration until a denaturing step is completed;
following the denaturing step, activating the infrared (IR) heating source at a second infrared wavelength selected so as to generate within the DNA fragment a second temperature for a second duration until an annealing step is completed; and
following the annealing step, activating the infrared (IR) heating source at a third infrared wavelength selected so as to generate within the DNA fragment a third temperature for a third duration until an extending step is completed;
wherein the first, second and third wavelengths generated by the IR source are selected to be coincident with corresponding absorptive wavelengths of the DNA fragment without being coincident with corresponding absorptive wavelengths of a fluid medium containing the DNA fragment so as to avoid so as to heat the DNA fragment without directly heating the fluid medium.

8. The method of claim 7, wherein an interior of the reaction chamber is initially maintained at an ambient temperature.

9. The method of claim 8, further comprising:
passing the DNA fragment through a first chamber containing a first infrared (IR) heating source therein, and activating the first infrared (IR) heating source at a first infrared wavelength so as to generate within the DNA fragment a first temperature for a first duration until the denaturing step is completed;
following the denaturing step, passing the DNA fragment through a second chamber containing a second infrared (IR) heating source therein, and activating the second infrared (IR) heating source at a second infrared wavelength so as to generate within the DNA fragment a second temperature for a second duration until the annealing step is completed; and
following the annealing step, passing the DNA fragment through a third chamber containing a third infrared (IR) heating source therein, and activating the third infrared (IR) heating source at a third infrared wavelength selected so as to generate within the DNA fragment a third temperature for a third duration until the extending step is completed.

10. The method of claim 9, wherein the DNA fragment is passed through the first second and third chambers by a conveyor.

11. The method of claim 7, wherein the fluid medium comprises water.

12. The method of claim 11, wherein the first, second and third wavelengths correspond to a frequency range of about 1000 cm⁻¹ to about 1200 cm⁻¹.
